(19) United States
(12) Patent Application Publication
(10) Pub. No.: US 2004/0068317 A1
(43) Pub. Date: Apr. 8, 2004

(54) Anterior chamber intraocular lens with size and position indicators

(76) Inventor: Patricia M. Knight, Laguna Niguel, CA (US)

Correspondence Address: Advanced Medical Optics, Inc., 1700 E. St. Andrew Place, Santa Ana, CA, 92705, US

(21) Appl. No.: 10/266,059

(22) Filed: Oct. 7, 2002

(51) Int. Cl. A61F 2/16

(52) U.S. Cl. 623/6.48

(57) ABSTRACT

The fixation members of an anterior chamber intraocular lens (IOL) are provided with indicators for aiding the sizing and placement of the IOL. In one embodiment, the indicators are lines, dots or other contrast based indicia formed at a predetermined distance away from the distal tips of the fixation members. In another embodiment, the indicators are colored zones extending a predetermined distance radially inwardly from the distal tips. The predetermined distance is selected such that the indicators are visible at the edge of a patient's cornea, even though the distal tips of the haptic members are hidden behind the scleral rim. The surgeon can evaluate whether the IOL is centered by checking that the indicators are symmetrical relative one another. Similarly, the surgeon can tell whether the IOL is properly sized by determining the distance between each line, or the edge of each colored zone, and the edge of the scleral rim, and comparing this distance to a desired value. Alternate embodiments include lines extending down haptic portions whereby the plurality of lines are arranged at predetermined spaced intervals provided on the fixation member or members.

### BACKGROUND OF THE INVENTION

This invention relates to intraocular lenses (IOLs). More particularly, the invention relates to IOLs placed in the anterior chambers of eyes, and to structures for aiding in the sizing and positioning of such IOLs.

Intraocular lenses (IOLs) are commonly used to modify or enhance vision. IOLs can be placed at various positions or locations within the eye. For example, IOLs can be placed in the anterior chamber of the eye, that is, the region posterior of the cornea and anterior of the iris.

Typical anterior chamber IOLs consist of an optic portion and one or more fixation members which are received in the iridio/corneal angle of the eye. The optic portion may be formed from either relatively rigid biocompatible materials such as polymethylacrylate (PMMA) or “soft” biocompatible materials such as silicone, certain acrylics and hydrogels. The fixation members may either be formed integrally with the optic portion, or separately formed and bonded to the optic portion using mechanical, thermal or chemical processes.

When implanted, both soft and rigid anterior chamber IOLS are subject to compressive forces which are exerted on the fixation members by the anterior chamber structure. These forces are to some extent desirable for centering and holding the lens in place. However, if excessive, the compressive forces can cause translational movement of the optic along the optical axis, which in turn can result in decentration of the optic and distortion of the visual images. In addition, such movement can cause the IOL to contact and damage the endothelial layer of the cornea. Excessive compression can also cause pupil retraction or ovalling, which is undesirable for both cosmetic and functional reasons.

In order to achieve the appropriate amount of compression of the anterior chamber IOL, it is critical that the IOL be correctly sized and properly placed in the anterior chamber angle. However, it is difficult to determine whether the size and position of an IOL is correct once it has been implanted, since the distal ends or “feet” of the fixation members are hidden behind the “white” or scleral rim surrounding the cornea.

Accordingly, it would be advantageous to provide anterior chamber IOLs with indicators for aiding in sizing and placement of the IOLs.

### SUMMARY OF THE INVENTION

Anterior chamber IOLs have been provided with indicator means for aiding in sizing and placement of the IOL. Specifically, the indicator means are in the form of visible indicators on the fixation members, or haptics, of the IOL.

In a first embodiment of the invention, an anterior chamber IOL comprises an optic body having a peripheral edge. A plurality of fixation members, or haptics, extend radially outwardly from the peripheral edge. Each fixation member has a distal tip that is configured to be received in the iridio/corneal angle of the anterior chamber and is hidden behind the scleral rim. A visible mark, such as a line, or a series of dots, or border portions of differently color zones, is provided on each fixation member at a predetermined distance from the distal tip. This distance is greater than the width of the scleral rim so that it can be observed by the surgeon after the IOL has been implanted. By confirming that this distance is within a predetermined range, and is the same for each fixation member, the surgeon can check whether the IOL is properly sized and centered.

In a second embodiment of the invention, the indicator means are in the form of a colored zone located at the distal end of each fixation member. By measuring the portion of each zone that extends beyond the scleral rim, the surgeon can determine whether the IOL has been appropriately sized and positioned, or if it has changed position.

Each and every feature described herein, and each and every combination of two or more of such features, is included within the scope of the present invention provided that the features included in such a combination are not mutually inconsistent.

These and other aspects and advantages of the present invention will become apparent in the following detailed description and claims, particularly when considered in conjunction with the accompanying drawings in which like parts bear like reference numerals. Likewise, those having a modicum of skill in the art are cognizant that the illustrations of preferred embodiments, and the claims appended hereto, are exemplary of the instant teachings, as opposed to limiting of the same.

### BRIEF DESCRIPTION OF THE DRAWINGS

FIG. 1 is a vertical sectional view of an eye and an exemplary anterior chamber intraocular lens (IOL) implanted therein;

FIG. 2 is a front plan view of a prior art anterior chamber IOL before implantation into the anterior chamber of an eye;

FIG. 3 is a front plan view of the prior art IOL after implantation into the anterior chamber of an eye;

FIG. 4 is a front plan view of an anterior chamber IOL according to embodiments of the present invention before implantation into the anterior chamber of an eye;

FIG. 5 is a front plan view of the IOL of FIG. 4 after implantation into the anterior chamber of an eye;

FIG. 6 is a front plan view of an anterior chamber IOL according to an alternate embodiment of the invention before implantation into the anterior chamber of an eye; and,

FIG. 7 is a front plan view of the IOL of FIG. 6 after implantation into the anterior chamber of an eye.

### DESCRIPTION OF THE PREFERRED EMBODIMENTS

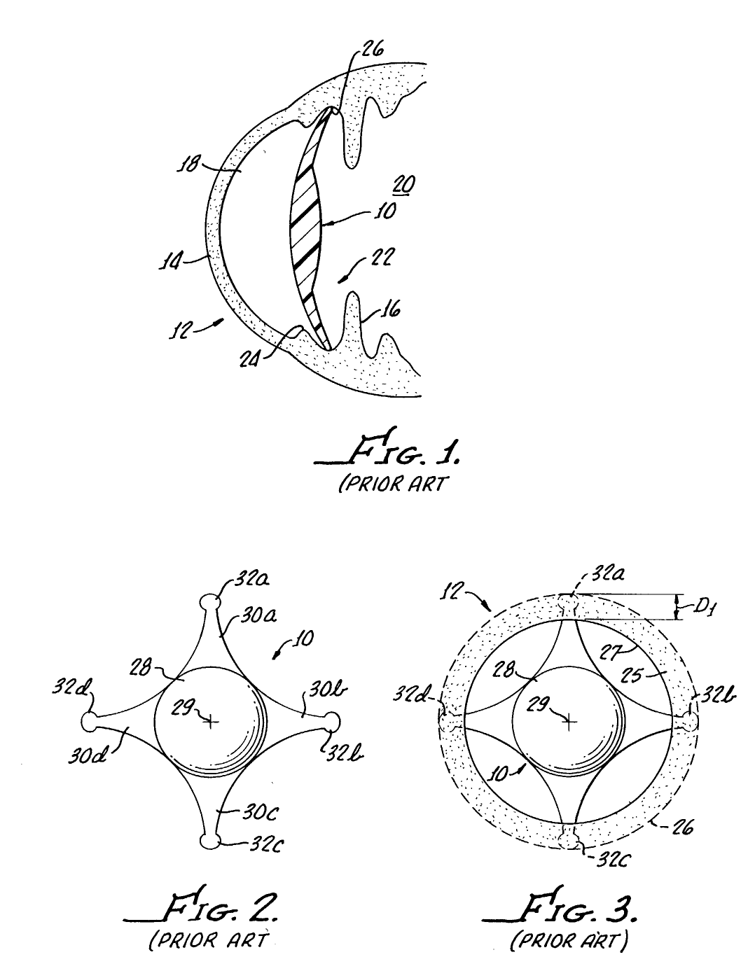

Referring now to FIG. 1, a prior art anterior chamber IOL 10 is shown implanted in an eye 12. The eye comprises a cornea 14 shown to the left or front of the eye and an annular iris 16 shown in the middle of the eye 12. The iris 16 divides the eye 12 into an anterior chamber 18 at the front of the eye and a posterior chamber 20 in back of the iris 16. The iris 16 also defines the aperture or pupil 22, which is a variable opening in the middle of the iris 16. In front of the iris 16 is the scleral spur 24. The scleral spur 24 and the iris 16 delimit the ciliary band or iridio/corneal angle 26.

The illustrated IOL 10, as best seen in FIG. 2, comprises an optic 28 having an optical axis 29, and a plurality of fixation members 30a-d. Each fixation member 30a-d has an enlarged distal tip or foot 32a-d that is configured to safely contact the ciliary band or angle 26. Other details regarding the structure and materials of the IOL 10 can be found in U.S. patent application Ser. No. 09/496,519, the disclosure of which is incorporated in its entirety by reference herein.

FIG. 3 shows the IOL 10 as it would be seen by a surgeon after implantation into an eye 12. From this vantage point, the distal tips 32a-d of the fixation members 30a-d are obscured by the scleral rim 25, which has an edge 27 located a distance D₁ radially inward of the ciliary band 26. The distance D₁ (i.e. the width of the scieral rim 25) may range from about 0.1 mm or less to about 0.6 mm or more, but is typically about 0.5 mm. In any case, because the distal tips 32a-d are obscured, it is difficult for the surgeon to determine whether the fixation members 30a-d are properly positioned in the ciliary band 26.

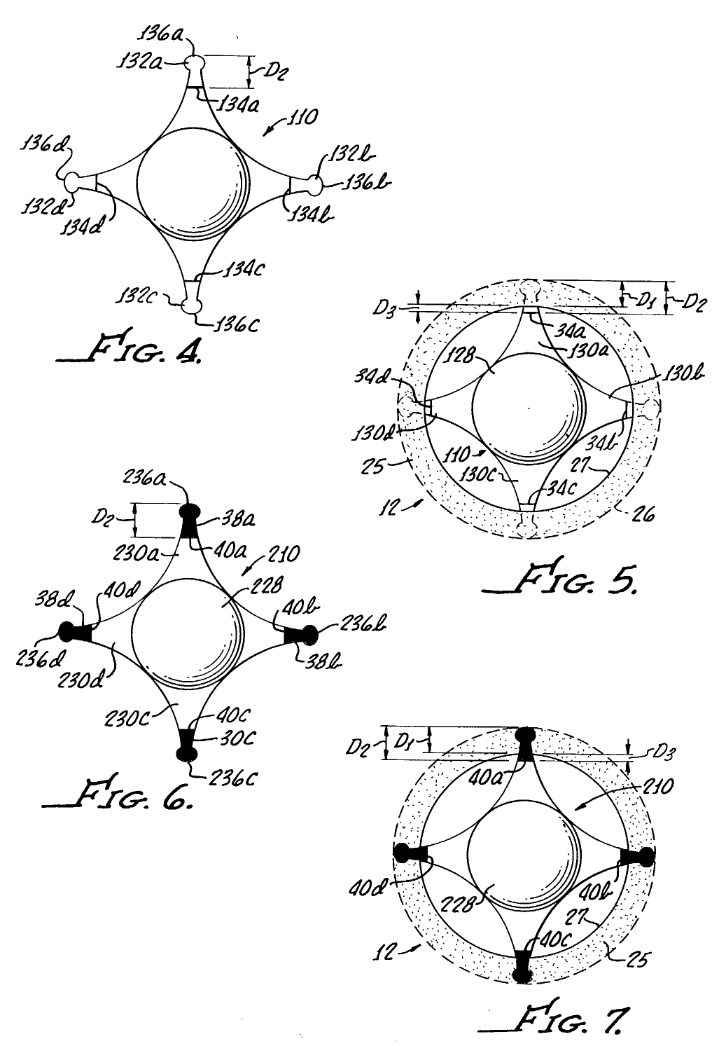

FIGS. 4 and 5 show an IOL 110 which is substantially similar to the IOL 10 of FIGS. 1-3, but provided with size and position indicia, or indicator means, according to the present invention. In this embodiment, the indicator means are in the form of visible marks or lines, for instance arcuate lines, 34a-d, formed on the fixation members 130a-d a predetermined distance D₂ from the distal edge 136a-d of each tip 132a-d. The distance D₂ is selected to be greater than the distance D₁ so that when the IOL 110 is correctly sized and positioned, the lines 34a-d will be clearly visible to the surgeon, as shown in FIG. 5. Preferably, D₂ is less than 1.0 mm and, more preferably, is in the range of about 0.2 to about 0.7 mm.

It is understood that the visible marks or lines may be readily substituted for by other indicia, such as a series of dots, which variable types of marking means are contemplated to be within the scop of the present invention. Likewise, plethoric other biocompatible marking systems including bioluminescent moieties may be used to practice the present invention, as will become clear to those skilled in the art.

If the IOL 110 is centered and properly fitted, the surgeon will see all the lines 34a-d a substantially fixed distance D₃ from the edge 27 of the scleral rim 25. If, on the other hand, the IOL 110 can be moved into an asymmetrical position wherein one or more of the lines 34a-d can not be seen, the surgeon will know that the IOL 110 is too small. Conversely, if the distance D₃ exceeds a desired maximum, the surgeon will know that the IOL 110 is too large. In either case, the IOL 110 can then be replaced by a better fitting one.

The marks or lines 34a-d may be formed on the fixation members 130a-d in a number of ways. For instance, each line 34a-d could be scribed into a fixation member 130a-d by a technique such as lathing, molding, or laser etching. Alternatively, the line could made by applying a dye of a different color than the rest of the fixation member. If the fixation members 130a-d are transparent, as is usually the case, any opaque color may be appropriate. However, if the fixation members 130a-d are themselves opaque, the color of the lines 34a-d should contrast that of the fixation members 34a-d.

Likewise, the present invention contemplates using indicator marks or lines in a ‘ruler-based’ format, whereby distances from portions of the haptics could be gauged, allowing for measurement, adjustment or gathering of more data by the surgeon.

An IOL 210 according to another embodiment of the invention is shown in FIGS. 6 and 7. With one exception, all the elements of IOL 210 are identical to the elements of IOL 110, and are indicated by the same reference numeral increased by 100. The exception is the indicator means, which are in the form of colored zones 38a-d, each of which extends radially inwardly from the distal edge 236 of its respective fixation member 23a-d to an inner boundary 40. The boundary 40 of each colored zone 38a-d serves a function similar to the line or mark 34 on IOL 110; that is, the distance D₂ from each distal edge 236a-d to its corresponding boundary 40a-d (i.e. the width of each colored zone 38) must be greater than the distance D₁ from the ciliary band 26 to the edge 27 of the scleral rim 25 so that a portion of each colored zone 38a-d is visible to the surgeon when the IOL 210 is correctly sized and positioned in the eye 12.

If the IOL 210 is centered and properly fitted, the visible portion of each colored zone 38a-d will be in the form of an arcuate band having a width D₃. This width D₃, which can either be measured with an instrument such as a keratoscope or approximated visually by an experienced surgeon, should be the same for each fixation member 230a-d. If, on the other hand, the IOL 210 can be moved into an asymmetrical position wherein some of the zones 38a-d are smaller than one another, or can not be seen at all, the surgeon will know that the IOL 210 is too small. Conversely, if the width D₃ exceeds a desired maximum for all the fixation members 230a-d, the surgeon will know that the IOL 210 is too large. If improperly sized, the IOL 210 can then be replaced by a better-fitting one.

The colored zones 38a-d may be formed by dyeing the ends of the fixation members 230a-d after manufacturing. Any opaque color may be selected if the fixation members 230a-d are transparent, or if the fixation members 230a-d are colored, a contrasting color should be selected.

Alternately, the colored zones 38a-d could be formed during the manufacturing process itself. For instance, in one method of manufacture, the colored zones 38a-d of the IOL 210 are formed by pouring a first precursor material, for instance a hydrogel-forming polymer, into a mold. A dye is added, and the mixture is polymerized. A hole is then bored in the polymerized material, and a clear or differently colored polymeric precursor material, which may be the same as or different from the first polymeric precursor material, is polymerized in the hole to form a button having a clear or first-colored inner portion and a second-colored annular periphery.

Optionally, another hole may be bored in the inner portion and a different polymeric precursor material polymerized in the hole to form a three-zoned button. In the case of a three-zoned button, the outer two zones are then lathed and milled as needed to form fixation members 230a-d, while the inner zone forms the optic 228. In a two-zoned button, the entire outer zone and an outer portion of the inner zone are lathed and milled to form the fixation members, while an inner portion of the inner zone would form the optic 228.

In another method of manufacture, a dye is added to a first polymeric precursor material, and the mixture is polymerized around a single rod or two concentric rods having the diameter desired of the optic portion plus the undyed or second-colored proximal portion of the fixation members 230a,b. If a single rod is used, this rod is removed and a clear or differently colored polymeric precursor material, either the same or different from the first polymeric material, is polymerized in the space left by the rod. If concentric rods are used, the rods may be removed serially, with the material for the proximal portion of the fixation members 230a,b being polymerized first in the space left by the outermost rod, and the material for the optic 228 being polymerized thereafter in the space left by the innermost rod. In either case, the resulting composite rod is then sliced to form buttons, each of which is then lathed and milled to form fixation members 230a,b and an optic 228 of an IOL 210.

Further details of manufacturing IOLs from buttons as discussed above can be found in U.S. Pat. No. 4,997,442 to Barrett, issued Mar. 5, 1991, and U.S. Pat. No. 5,217,491, to Vanderbilt, the disclosures of both of which are incorporated in entirety by reference herein.

While this invention has been described with respect to various specific examples and embodiments, it is to be understood that the invention is not limited thereto and that it can be variously practiced within the scope of the claims. For example, the size and position indicators of the present invention are not restricted to use with fixation members of the configurations shown in FIGS. 1-7, but can be applied to fixation members of variety of different sizes, shapes and material. Similarly, the number of fixation members, as well as the sizes, shapes and methods of forming the indicator marks or zones are merely exemplary and not intended to be limiting.

What is claimed is:

1. An intraocular lens for implantation in an eye, the intraocular lens comprising:
an optic having a peripheral edge;
at least one fixation member having a proximal end secured to the optic and a distal end extending a predetermined length from the peripheral edge of the optic;
indicator means on the at least one fixation member for aiding in sizing and placement of the intraocular lens.

2. The intraocular lens of claim 1 wherein:
the intraocular lens is configured for placement in an anterior chamber of the eye, the eye including a scleral rim extending a first distance into the anterior chamber;
the distal end of the at least one fixation member is configured to be received in a ciliary band located behind the scieral rim, the distal end including a tip portion obscured by scleral rim when the intraocular lens is implanted in the eye; and
the indicator means comprise at least one visible indicator located radially inward of the scleral rim when the intraocular lens is implanted in the eye.

3. The intraocular lens of claim 1, wherein:
the distal end includes a distal tip; and
the indicator means comprise at least one visible mark located radially inwardly from the distal tip.

4. The intraocular lens of claim 3, wherein the visible mark comprises a line provided on the fixation member.

5. The intraocular lens of claim 3, wherein the visible mark is scribed on the fixation member.

6. The intraocular lens of claim 3, wherein the fixation member is substantially transparent and the visible mark is substantially opaque.

7. The intraocular lens of claim 3, wherein the fixation member is colored and wherein the visible mark is a different color from the fixation member.

8. The intraocular lens of claim 1, wherein:
the distal end includes a distal tip; and
the indicator means comprise a visible zone extending radially inwardly from the distal tip.

9. The intraocular lens of claim 8, wherein the visible zone extends inwardly a distance less than the length of the fixation member.

10. The intraocular lens of claim 8, wherein the fixation member is substantially transparent and the visible zone is substantially opaque.

11. The intraocular lens of claim 8, wherein the fixation member is colored and wherein the visible zone is a different color from the fixation member.

12. The intraocular lens of claim 2, wherein:
the distal end includes a distal tip; and
the indicator means comprise a visible mark located a second distance from the distal tip.

13. The intraocular lens of claim 12, wherein the second distance is greater than the first distance.

14. The intraocular lens of claim 2, wherein:
the distal end includes a distal tip; and
the indicator means comprise a visible zone extending a second radial distance inwardly from the distal tip.

15. The intraocular lens of claim 14, wherein the second radial distance is greater than the first radial distance and less than the length of the fixation member.

16. The intraocular lens of claim 1, wherein:
the at least one fixation member includes a plurality of fixation members; and
the indicator means comprise an indicator provided on each of the fixation members.

17. The intraocular lens of claim 16, wherein:
each of the fixation members is of equal length and includes a distal tip; and
each indicator is a visible mark located an equal radial distance from the distal tip.

18. The intraocular lens of claim 16, wherein:
each of the fixation members is of equal length and includes a distal tip; and
each indicator is a visible zone extending an equal radial distance inwardly from the distal tip.

19. A method of evaluating the fit of an intraocular lens in an eye, comprising:
placing an intraocular lens behind a structure extending a first radial distance into the eye, the intraocular lens including an optic having a peripheral edge and at least one fixation member, the fixation member including a distal tip that is obscured by the structure when placed in the eye, and at least one indicator located a second radial distance away from the distal tip, the second distance being greater than the first distance when the fixation member is uncompressed;
determining a third radial distance between the indicator and an end of the structure; and
comparing the third radial distance to a desired radial distance.

20. The method of claim 19, further comprising a step of replacing the intraocular lens with a different sized intraocular lens if the third radial distance differs significantly from the desired radial distance.

21. The method of claim 20, wherein:
the intraocular lens is configured to be implanted in an anterior chamber of the eye;
the distal tip of the fixation member is configured to be positioned behind a scleral rim having a width; and
the first radial distance is the width of the scleral rim.

22. The method of claim 19, wherein the indicator is a line provided on the fixation member.

23. The method of claim 19, further comprising a colored zone extending radially inwardly from the distal tip of the fixation member, wherein the indicator is a proximal edge of the colored zone.

24. A method of evaluating the position of an intraocular lens in an eye, comprising:
placing an intraocular lens behind a structure projecting into the eye, the intraocular lens including a plurality of fixation members, each fixation member having a distal tip and an indicator located radially inwardly of an end of the structure when the lens properly fitted in the eye;
determining a distance between the indicator and an end of the structure for each fixation member; and
determining whether this distance is substantially equal for all fixation members.

25. The method of claim 24, wherein:
the intraocular lens is configured to be implanted in an anterior chamber of the eye;
the structure is a scleral rim; and
the first radial distance is the width of the scleral rim.

26. The method of claim 24, wherein the indicator is a line provided on the fixation member.

27. The method of claim 24, further comprising a colored zone extending radially inwardly from the distal tip of the fixation member, wherein the indicator is a proximal edge of the colored zone.

28. The intraocular lens of claim 3, wherein the visible mark comprises a series of dots provided on the fixation member.

29. The intraocular lens of claim 3, wherein the visible mark comprises a plurality of lines at predetermined spaced intervals provided on the fixation member.

30. The method of claim 19, wherein the indicator is a series of dots provided on the fixation member.

31. The method of claim 19, wherein the indicator comprises a plurality of lines at predetermined spaced intervals provided on the fixation member.

32. The method of claim 24, wherein the indicator is a series of dots provided on the fixation member.

33. The method of claim 24, wherein the indicator comprises a plurality of lines at predetermined spaced intervals provided on the fixation member.
